(19) United States
(12) Patent Application Publication
(10) Pub. No.: US 2021/0275340 A1
(43) Pub. Date: Sep. 9, 2021

(54) Neck Brace and Mouth Piece That Permit a Range of Movement While Maintaining an Open Airway

(71) Applicant: Sanford Health, Sioux Falls, SD (US)

(72) Inventors: Michael Sand, Sioux Falls, SD (US); Patrick W. Kelly, Sioux Falls, SD (US)

(21) Appl. No.: 17/260,084

(22) PCT Filed: Jul. 17, 2019
(86) PCT No.: PCT/US2019/042171

§ 371 Date: Jan. 13, 2021

Related U.S. Application Data
(60) Provisional application No. 62/699,218, filed on Jul. 17, 2018.

(51) Int. Cl. A61F 5/058; A61F 5/56

(52) U.S. Cl. CPC A61F 5/05883; A61F 5/566

(57) ABSTRACT

The present disclosure provides a device that includes (a) a chest support, (h) a neck support, (c) a chin support, (d) a pair of upper side struts each having a first end coupled to the chin support and a second end coupled to the neck support, and (e) a pair of lower side struts each having a first end coupled to the chest support and a second end coupled to the neck support.

### RELATED APPLICATIONS

This application claims the benefit of priority to U.S. Provisional Application No. 62/699,218 entitled “Neck Brace and Mouth Piece That Permit a Range of Movement While Maintaining an Open Airway,” filed on Jul. 17, 2018, the contents of which are hereby incorporated by reference in their entirety.

### BACKGROUND

Obstructive sleep apnea affects approximately 42 million American adults. Current treatments for sleep apnea include continuous positive airway pressure (CPAP). CPAP is a form of positive airway pressure ventilator, which applies mild air pressure on a continuous basis to keep the airways continuously open in individuals with sleep apnea. However, there are poor compliance statistics with CPAP devices. Many patients abandon CPAP treatment after using it for a time, and many patients never commence therapy after initial diagnosis and CPAP titration. CPAP devices are traditionally large and bulky, and many individuals find them uncomfortable to sleep with. Therefore, an improved device and corresponding method of use may be desirable.

### SUMMARY

The present disclosure relates to an improved neck brace and mouth piece that permit a range of movement while maintaining an open airway.

Thus, in a first aspect, the present invention provides a device that includes (a) a chest support, (b) a neck support, (c) a chin support, (d) a pair of upper side struts each having a first end coupled to the chin support and a second end coupled to the neck support, and (e) a pair of lower side struts each having a first end coupled to the chest support and a second end coupled to the neck support.

In a second aspect, the present invention provides a device that includes (a) a chest support, (b) a neck support having a first upper end and a second lower end, (c) a pair of upper side struts each having a first end coupled to the chest support and a second end coupled to the first upper end of the neck support, and (d) a pair of lower side struts each having a first end coupled to the chest support and a second end coupled to the second lower end of the neck support.

In a third aspect, the present invention provides a method that includes (a) positioning the device of the first aspect on a user, such that the chest support is positioned on a chest of the user, the neck support is positioned on a posterior of a neck of the user, and the chin support is positioned on or under a chin of the user, and (b) adjusting a length of the first positioning strut such that the chin of the user is at a desired angle with respect to the chest of the user.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a side view of a device, according to an example embodiment.

FIG. 2 illustrates a front view of the device of FIG. 1, according to an example embodiment.

FIG. 3 illustrates a first positioning strut of the device of FIG. 1, according to an example embodiment.

FIG. 4 illustrates a front view of another example device, according to an example embodiment.

FIG. 5 illustrates a side view of another device, according to an example embodiment.

FIG. 6 is a flow chart depicting functions that can be carried out in accordance with example embodiments of the disclosed methods.

### DETAILED DESCRIPTION OF THE INVENTION

Exemplary devices and methods are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. One of ordinary skill in the art will readily understand that certain aspects of the disclosed systems and methods can be arranged and combined in a wide variety of different configurations, all of which are contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment a include elements that are not illustrated in the Figures.

As used herein, with respect to measurements, “about” means +/−5%.

As used herein, “coronal plane” means a plane that divides the body into back and front portions.

As used herein, “sagittal plane” means a plane that is parallel to the sagittal suture, dividing the body into left and right portions.

As used herein, “axial plane” means a plane that divides the body into top and bottom portions.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, device, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, device, structure, article, element, component, or hardware which enable the system, apparatus, device, structure, article, element, component, or hardware to perform the specified function without further modification. For purposes of this disclosure, a system, apparatus, device, structure, article, element, component, or hardware described as being “configured to” perform a particular function may additionally or alternatively be described as being “adapted to” and/or as being “operative to” perform that function.

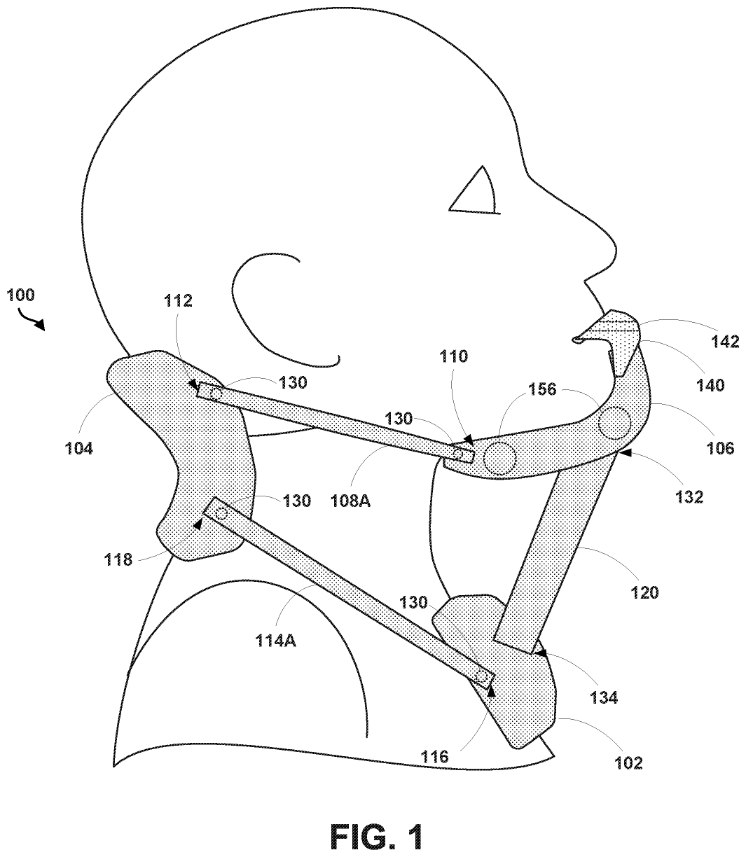

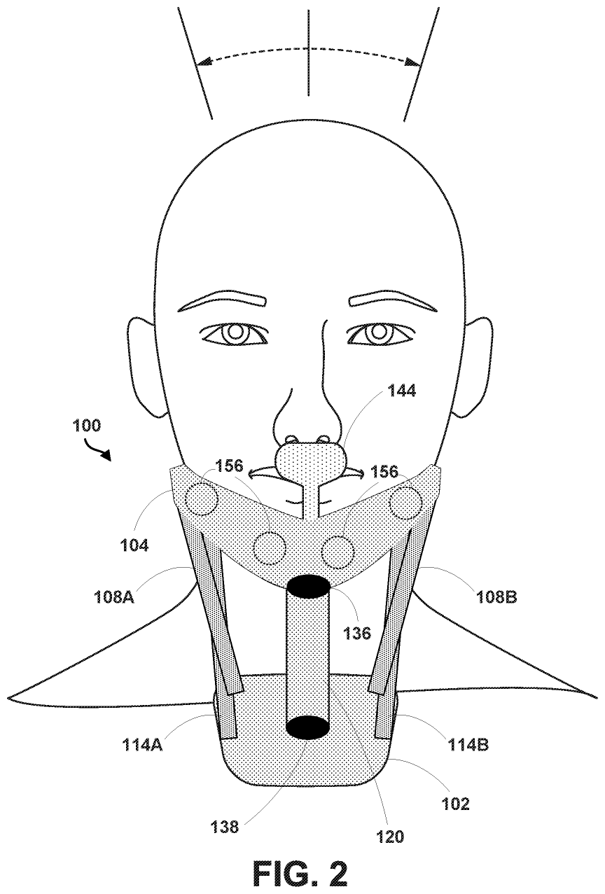

With reference to the Figures, FIG. 1 illustrates a device 100, according to an example embodiment. As shown in FIG. 1, the device 100 includes a chest support 102, a neck support 104, and a chin support 106. As shown in the front view of the device 100 in FIG. 2, the device 100 further includes a pair of upper side struts 108A, 108B each having a first end 110 coupled to the chin support 106 and a second end 112 coupled to the neck support 104. The device 100 further includes a pair of lower side struts 114A, 114B each having a first end 116 coupled to the chest support 102 and a second end 118 coupled to the neck support 104.

In one example, the device 100 further includes a first positioning strut 120 having a first end 132 coupled to the chin support 106 and a second end 134 coupled to the chest support 102. A length of the first positioning strut 120 is adjustable. In particular, the length of the first positioning strut 120 is adjustable between about 5 cm to about 25 cm. The mechanism for adjusting the length of the first positioning strut 120 may take a variety of forms. In one example, the first positioning strut 120 comprises a scissor lift mechanism including one or more pivot points about which portions of the first positioning strut 120 are configured to rotate to thereby set the adjustable length of the first positioning strut 120

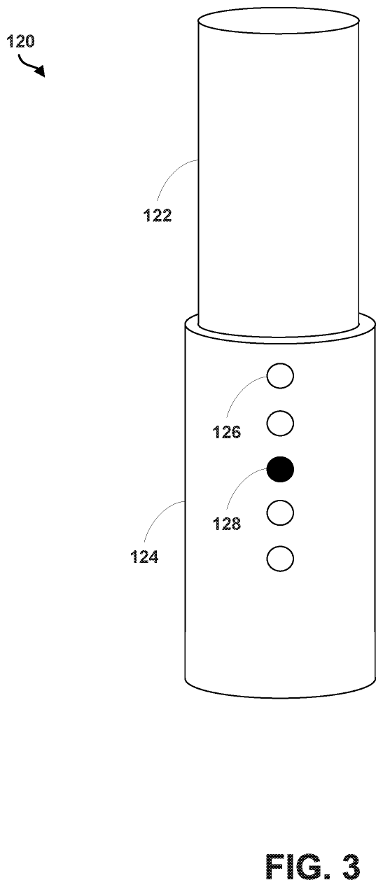

In another example, as shown in FIG. 3, the first positioning strut 120 comprises an inner tube 122 arranged within an outer tuber 124 such that the inner tube 122 and outer tube are configured to move relative to each other from a first position to a second position to thereby adjust the length of the first positioning strut 120. In such an example, the device 100 may further include a plurality of through-holes 126 arranged in series along at least a portion of the length of the outer tube 124, and a spring-loaded button 128 coupled to the inner tube 122 and disposed through one of the plurality of through-holes 126 in the first position. The spring-loaded button 128 may be configured to be depressed such that the inner tube 122 and outer tube 124 move relative to each other to the second position in which the spring-loaded button 128 is disposed through another of the plurality of through-holes 126 to thereby set the adjustable length of the first positioning strut 120.

In another example, the inner tube 122 may include a projection configured to fit within a track in the outer tube 124 such that the inner tube 122 and outer tube 124 are configured to move relative to each other from a first position to a second position to thereby adjust the length of the first positioning strut 120. In an alternative example, the outer tube 124 may include a projection configured to fit within a track in the inner tube 122 such that the inner tube 122 and outer tube 124 are configured to move relative to each other from a first position to a second position to thereby adjust the length of the first positioning strut 120. In such examples, a clamp or other locking mechanism may be used to lock the inner tube 122 with respect to the outer tube 124 to prevent relative movement between the two components once the desired length of the first positioning strut 120 is achieved.

In another example, a gear system, such as a rack and pinion gear system, may be used to enable the inner tube 122 and outer tube 124 to move relative to each other from a first position to a second position to thereby adjust the length of the first positioning strut 120. In yet another embodiment, a surface of the inner tube 122 may be threaded, and a corresponding surface on the outer tube 124 may also be threaded such that a rotation of the inner tube 122 with respect to the outer tube 124 (or similarly a rotation of the outer tube 124 with respect to the inner tube 122) is configured to move the inner tube 122 and/or the outer tube 124 relative to each other from a first position to a second position to thereby adjust the length of the first positioning strut 120. Although only two tubes are discussed in the examples above, the first positioning strut 120 may include three or more telescoping tubes to achieve the adjustability described above. Alternative adjustable length mechanisms are possible as well.

In one embodiment, one or more of the first positioning strut 120, the pair of upper side struts 108A, 108B, and the pair of lower side struts 114A, 114B are removably coupled to one or more of the chest support 102, the neck support 104, and the chin support 106. In one particular example, one or more of the first positioning strut 120, the pair of upper side struts 108A, 108B, and the pair of lower side struts comprise at least one magnet 130 to removably couple a given strut to one or more of the chest support 102, the neck support 104, and the chin support 106. Other removable coupling mechanisms are possible as well.

In one embodiment, a first end 132 of the first positioning strut 120 is movably coupled to the chin support 106, and a second end 134 of the first positioning strut 120 is movably coupled to the chest support 102. Such an arrangement may enable user comfort when wearing the device 100 during sleep. In one particular example, the first end 132 of the first positioning strut 120 is rotatably coupled to the chin support 106 via a first joint 136 having at least one degree of freedom, and the second end 134 of the first positioning strut 120 is rotatably coupled to the chest support 102 via a second joint 138 having at least one degree of freedom. The first joint 136 and the second joint 138 may take a variety of forms, including one of a ball and socket joint, a pivot joint, a combination of two or more struts configured to scissor, or a living hinge, as non-limiting examples. The living hinge may take the form of a flexible polymer molded over and surrounding each the first end 132 and the second end 134 of the first positioning strut 120, such that the polymer has a base attachable to the chin support 106 and chest support 102 permitting flexion at the respective joints.

In another embodiment, however, the first end 132 of the first positioning strut 120 is fixedly coupled or locked to the chin support 106, and the second end 134 of the first positioning strut 120 is fixedly coupled or locked to the chest support 102. Such an arrangement prevents movement of the head of the user in one or more of the coronal plane, sagittal plane, and axial plane.

In one embodiment, a length of at least one strut of the pair of upper side struts 108A, 108B and the pair of lower side struts 114A, 114B has an adjustable length in one or more of the coronal, sagittal, and axial planes. Such an arrangement may beneficially enable a one size fits all option such that the lengths of the pair of upper side struts 108A, 108B and/or the pair of lower side struts 114A, 114B can be adjusted by the user to more comfortably fit the user when in use. In such an example, the at least one strut of the pair of upper side struts 108A, 108B and the pair of lower side struts 114A, 114B may include an inner tube arranged within an outer tuber such that the inner tube and outer tube are configured to move relative to each other from a first position to a second position to thereby adjust the length of the at least one strut. As such, the adjustable length of the at least one strut of the pair of upper side struts 108A, 108B and the pair of lower side struts 114A, 114B may include any of the adjustable length mechanisms of the first positioning strut 120 described above, as well as any other alternative adjustable length mechanisms. In another example, the at least one strut of the pair of upper side struts 108A, 108B and the pair of lower side struts 114A, 114B may include adjustable chords or straps that pull the chest support 102 and/or chin support 106, which are set at a fixed distanced from one other based on the non-adjustable pair of side struts, toward the neck support thereby causing the extension of the head in the sagittal plane.

The device 100 may further include an oral appliance 140 coupled to the chin support 106. The oral appliance 140 is configured to adjust a jaw thrust motion in relation to a maxillary of the user. As such, the oral appliance 140 may have a degree of freedom allowing for the opening of the jaw and mouth. Such oral appliances 140 may thereby change the dynamics of the jaw to affect the opening of the airway. The oral appliance 140 may include at least one through-hole 142 configured to receive one or more oral airway maintenance devices. In particular examples, the oral appliance 140 may be an oropharyngeal airway or an oral airway intubation device. The oral appliance 140 may have channels running along the sides of the appliance that allow air through, and/or air may flow through the at least one through-hole 142 in the center of the oral appliance 140. Such oral appliances 140 may assist in holding the tongue of a patient in place, and may further provide an additional air passage.

As shown in FIG. 2, the device 100 may further include a nasal appliance 144 coupled to the chin support 106. In another example, the nasal appliance 144 may be coupled to the oral appliance 140, such that the device 100 includes both a nasal appliance 144 and an oral appliance 140. In particular examples, the nasal appliance 144 may be a nasopharyngeal airway or nasal trumpet airway tube. The nasal appliance 144 may include at least one through-hole. Such a nasal appliance 144 may be designed to be inserted into the nasal passageway to secure an open airway.

The device 100 may further include an endotracheal appliance coupled to chin support 106. Such an endotracheal appliance may comprise an endotracheal intubation technique for use in situations where an opening is required through the trachea of the patient to maintain an open airway or to serve as a conduit through which to administer certain drugs.

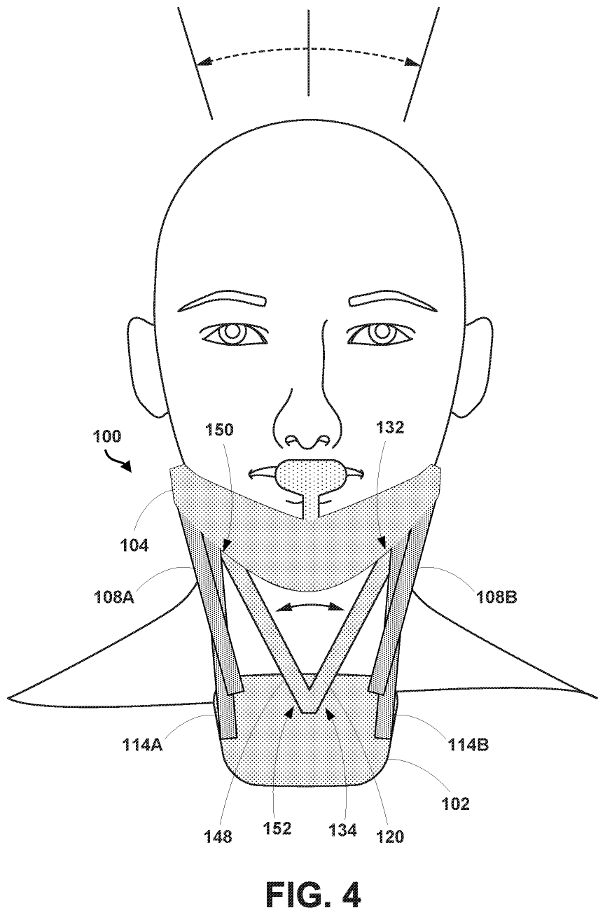

In one embodiment, as shown in FIG. 4, the device 100 may further include a second positioning strut 148 having a first end 150 and a second end 152. In such an example, the first end 132 of the first positioning strut 120 is coupled to a first side of the chin support 106, the first end 150 of the second positioning strut 148 is coupled to a second side of the chin support 106, and the second end 152 of the second positioning strut 148 is coupled to the chest support 102. In one example, the length of the first positioning strut 120 and the second positioning strut 148 are fixed to limit movement in the coronal plane. In another example, the length of the first positioning strut 120 and/or the second positioning strut 148 may be adjustable to enable movement in the coronal plane. In such an example, the first positioning strut 120 and/or the second positioning strut 148 may include any of the adjustable length mechanisms described above, as well as any other alternative adjustable length mechanisms. In another example, the second positioning strut 148 may combine with the first positioning strut 120 to form a scissor mechanism with one or more pivots about which portions of the first positioning strut 120 and/or portions of the second positioning strut 148 are configured to rotate to thereby set the adjustable length of the scissor mechanism.

In one example, the first positioning strut 120 and the pair of lower side struts 114A, 114B each include a rigid material, and the pair of upper side struts 108A, 108B each include a flexible material. In another example, the first positioning strut 120 and the pair of upper side struts 108A, 108B each include a rigid material, and the pair of lower side struts 114A, 114B each include a flexible material. In another example, each of the pair of lower side struts 114A, 114B and the pair of upper side struts 108A, 18B include a flexible material. In yet another example, each of the first positioning strut 120, the pair of lower side struts 114A, 114B, and the pair of upper side struts 108A, 108B each include a rigid material.

In one particular example, the pair of lower side struts 114A, 114B and/or the pair of upper side struts 108A, 108B comprise a rigid plastic. In another example, the pair of lower side struts 114A, 114B and/or the pair of upper side struts 108A, 108B comprise a cord. The first positioning strut 120 may include a compressive material such that one or more components of the first positioning strut 120 can stretch or compress independent of each other (i.e., a tilted head where one component of the first positioning strut 120 has lengthened). As discussed above, the first positioning strut 120 may comprise a scissor lift mechanism to enable up and down motions (i.e., the user is nodding). Further, the second end 134 of the first positioning strut 120 may include a pivot 154 to enable side to side twisting motion (i.e., the user is shaking their head “no”). Such an arrangement may help ensure user comfort when the user is sleeping with their head twisted to the side.

In one example, the device 100 further includes one or more electrodes 156 positioned on the chin support 106. In one such example, the one or more electrodes 156 are positioned on the chin support 106 such that the one or more electrodes 156 are configured to contact a throat of a user when in use. In another example, the one or more electrodes 156 are positioned on the chin support 106 such that the one or more electrodes 156 are configured to contact a jaw of a user when in use. In both examples described above, the one or more electrodes 156 may provide low grade, transcutaneous electrical nerve stimulation on the contact points of the jaw and throat of the user to allow stimulation of muscles in those regions to keep the tongue of the user from obstructing the airway. In particular, the one or more electrodes 156 on the throat of the user may provide transcutaneous pacing of the throat muscles to prevent the tongue of the user from obstructing the airway. The one or more electrodes 156 on the jaw of the user may increase muscle tone to keep the tongue of the user from dropping back and obstructing the airway. Additional locations for the one or more electrodes 156 are possible as well.

In use, the chest support 102 is positioned on a chest of the user, the neck support 104 is positioned on a posterior of a neck of the user, and the chin support 106 is positioned on or under a chin of the user. The material for the chest support 102, the neck support 104, and the chin support 106 may be the same rigid material as one or more of the pair of upper side struts 108A, 108B, the pair of lower side struts 114A, 114B, and the first positioning strut 120 since the chest support 102, the neck support 104, and the chin support 106 will remain relatively stationary when the device 100 is in use. The material for the chest support 102, the neck support 104, and the chin support 106 may be the same as one another, or each component may comprise a different material. However, since these components will directly contact the user, the chest support 102, the neck support 104, and the chin support 106 may have a padded region to ensure user comfort when in use. For example, one surface of the chest support 102, the neck support 104, and the chin support 106 may be padded to conform to the shape of the user. Such padding may be any appropriate cushioning material, such as foam or rubber as non-limiting examples.

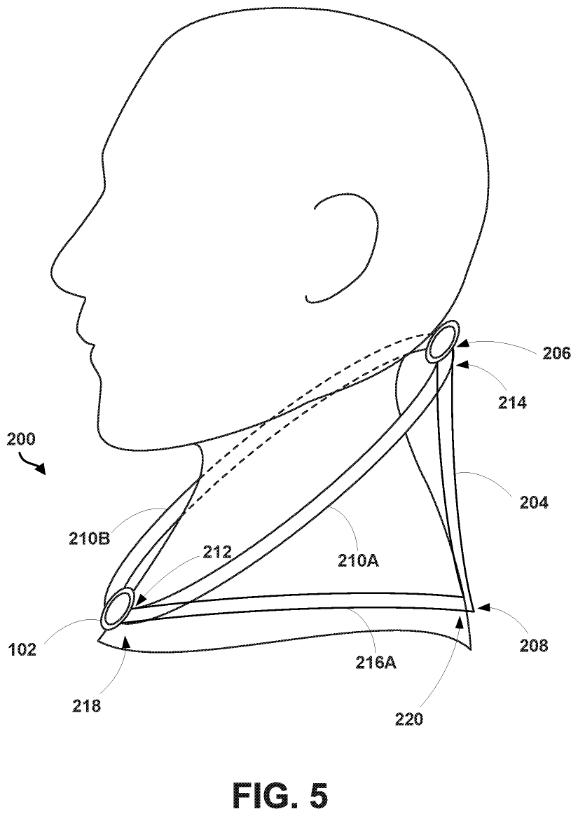

FIG. 5 illustrates another device 200, according to an example embodiment. As shown in FIG. 5, the device 200 includes a chest support 202, and a neck support 204 having a first upper end 206 and a second lower end 208. The device 200 also includes a pair of upper side struts 210A, 210B each having a first end 212 coupled to the chest support 202 and a second end 214 coupled to the first upper end 206 of the neck support 204. The pair of upper side struts 210A, 210B may each comprise a spring configured to bias the neck support 204 away from the chest support 202. The device 200 also includes a pair of lower side struts 216A, 216B each having a first end 218 coupled to the chest support 202 and a second end 220 coupled to the second lower end 208 of the neck support 204.

In one example, the pair of upper side struts 210A, 210B and the pair of lower side struts 216A, 216B each include a flexible material. In another example, the pair of upper side struts 210A, 210B each include a rigid material, and the pair of lower side struts 216A, 216B each include a flexible material. In yet another example, the pair of upper side struts 210A, 210B each include a flexible material, and the pair of lower side struts 216A, 216B each include a rigid material.

In use, the chest support 202 is positioned on a chest of the user, and the neck support 204 is positioned on a posterior of a neck of the user. The material for the chest support 202 and the neck support 204 may be the same rigid material as one or more of the pair of upper side struts 210A, 210B and the pair of lower side struts 216A, 216B since the chest support 202 and the neck support 204 will remain relatively stationary when the device 100 is in use. The material for the chest support 202 and the neck support 204 may be the same as one another, or each component may comprise a different material. However, since these components will directly contact the user, the chest support 202 and the neck support 204 may have a padded region to ensure user comfort when in use. For example, one surface of the chest support 202 and the neck support 204 may be padded to conform to the shape of the user. Such padding may be any appropriate cushioning material, such as foam or rubber as non-limiting examples.

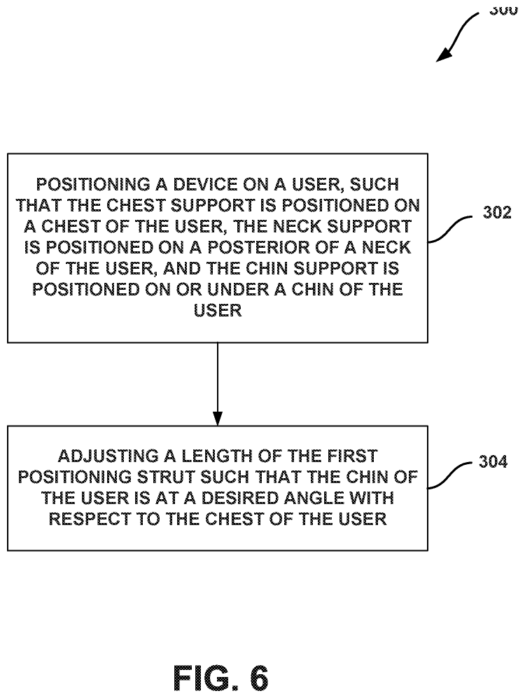

FIG. 6 is a block diagram of an example method for maintaining a chin of a user is at a desired angle with respect to a chest of the user. Method 300 shown in FIG. 6 presents an embodiment of a method that could be used by the device 100 of FIGS. 1-5, as described above. Method 300 may include one or more operations, functions, or actions as illustrated by one or more of blocks 302-304. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

Initially, at block 302, the method 300 includes positioning the device of any of the embodiments described above in relation to FIGS. 1-4 on a user, such that the chest support 102 is positioned on a chest of the user, the neck support 104 is positioned on a posterior of a neck of the user, and the chin support 106 is positioned on or under a chin of the user. At block 304, the method 300 includes adjusting a length of the first positioning strut 120 such that the chin of the user is at a desired angle with respect to the chest of the user.

The method 300 may further include positioning an oral appliance 140 in a mouth of the user prior to adjusting the length of the first positioning strut 120. As used herein, the “chest” includes the sternum, collar bone, and ribs of the user. In one example, the desired angle ranges from about 0 degrees to about 45 degrees. The desired angle may be selected to maintain an open airway, thereby preventing sleep apnea in the user. In another example, the desired angle helps maintain an open airway for pre- and post-anesthesia along with use in emergency room and ambulance transport.

The method 300 may further include stimulating a throat of the user via one or more electrodes positioned on the chin support 106 to thereby stimulate one or more muscles of the user to prevent a tongue of the user from obstructing an airway of the user. In another example, the method 300 may further include stimulating a jaw of the user via one or more electrodes positioned on the chin support to thereby stimulate one or more muscles of the user to prevent a tongue of the user from obstructing an airway of the user.

Another example method includes positioning the device of any of the embodiments described above in relation to FIG. 5 on a user, such that the chest support 202 is positioned on a chest of the user and the neck support 204 is positioned on a posterior of a neck of the user.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the invention can be combined unless the context clearly dictates otherwise. The various aspects And embodiments disclosed herein are for purposes of illustration and are not intended to be limiting, with the true scope and spirit being indicated by the claims.

1. A device comprising:
a chest support;
a neck support;
a chin support;
a pair of upper side struts each having a first end coupled to the chin support and a second end coupled to the neck support; and
a pair of lower side struts each having a first end coupled to the chest support and a second end coupled to the neck support.

2. The device of claim 1, further comprising:
a first positioning strut having a first end coupled to the chin support and a second end coupled to the chest support.

3. The device of claim 2, wherein the length of the first positioning strut is adjustable between about 5 cm to about 25 cm.

4. The device of claim 2, wherein one or more of the first positioning strut, the pair of upper side struts, and the pair of lower side struts are removably coupled to one or more of the chest support, the neck support, and the chin support.

5. The device of any one of claims 2-4 claim 2, wherein the first end of the first positioning strut is movably coupled to the chin support, and wherein the second end of the first positioning strut is movably coupled to the chest support.

6. The device of claim 2, wherein the first end of the first positioning strut is rotatably coupled to the chin support via a first joint having at least one degree of freedom, and wherein the second end of the first positioning strut is rotatably coupled to the chest support via a second joint having at least one degree of freedom.

7. The device of claim 6, wherein the first joint and the second joint each comprise one of a ball and socket joint, a pivot joint, a combination of two or more struts configured to scissor, or a living hinge.

8. The device of claim 2, wherein the first positioning strut comprises an inner tube arranged within an outer tuber such that the inner tube and outer tube are configured to move relative to each other from a first position to a second position to thereby adjust the length of the first positioning strut.

9. The device of claim 8, further comprising:
a plurality of through-holes arranged in series along at least a portion of the length of the outer tube; and
a spring-loaded button coupled to the inner tube and disposed through one of the plurality of through-holes in the first position, wherein the spring-loaded button is configured to be depressed such that the inner tube and outer tube move relative to each other to the second position in which the spring-loaded button is disposed through another of the plurality of through-holes to thereby set the adjustable length of the first positioning strut.

10. The device of claim 2, further comprising:
a second positioning strut having a first end and a second end, wherein the first end of the first positioning strut is coupled to a first side of the chin support, wherein the first end of the second positioning strut is coupled to a second side of the chin support, and wherein the second end of the second positioning strut is coupled to the chest support.

11. The device of claim 2, wherein one or more of the first positioning strut, the pair of upper side struts, and the pair of lower side struts comprise at least one magnet to removably couple a given strut to one or more of the chest support, the neck support, and the chin support.

12. The device of claim 2, wherein the first positioning strut and the pair of lower side struts each comprise a rigid material, and wherein the pair of upper side struts each comprise a flexible material.

13. The device of claim 1, wherein a length of at least one strut of the pair of upper side struts and the pair of lower side struts has an adjustable length in one or more of a coronal plane, a sagittal plane, and an axial plane.

14. The device of claim 13, wherein the at least one strut comprises an inner tube arranged within an outer tuber such that the inner tube and the outer tube of the at least one strut are configured to move relative to each other from a first position to a second position to thereby adjust the length of the at least one strut.

15. The device of claim 1, further comprising an oral appliance coupled to the chin support, wherein the oral appliance is configured to adjust a jaw thrust motion in relation to a maxillary of a user.

16. The device of claim 15, wherein the oral appliance includes at least one through-hole configured to receive one or more oral airway maintenance devices.

17. (canceled).

18. The device of claim 1, further comprising:
one or more electrodes positioned on the chin support.

19. The device of claim 18, wherein the one or more electrodes are positioned on the chin support such that the one or more electrodes are configured to contact a throat of a user when in use.

20. The device of claim 18, wherein the one or more electrodes are positioned on the chin support such that the one or more electrodes are configured to contact a jaw of a user when in use.

21. (canceled).

22. (canceled).

23. A method comprising:
positioning the device of claim 1 on a user, such that the chest support is positioned on a chest of the user, the neck support is positioned on a posterior of a neck of the user, and the chin support is positioned on or under a chin of the user; and
adjusting a length of the first positioning strut such that the chin of the user is at a desired angle with respect to the chest of the user.

24. The method of claim 23, wherein the desired angle ranges from about 0 degrees to about 45 degrees.

25. The method of claim 23, wherein the desired angle maintains an open airway thereby preventing sleep apnea in the user.

26. The method of claim 23, further comprising:
positioning an oral appliance in a mouth of the user prior to adjusting the length of the first positioning strut.

27. The method of claim 23, further comprising
stimulating a throat of the user via one or more electrodes positioned on the chin support to thereby stimulate one or more muscles of the user to prevent a tongue of the user from obstructing an airway of the user.

28. The method of claim 23, further comprising
stimulating a jaw of the user via one or more electrodes positioned on the chin support to thereby stimulate one or more muscles of the user to prevent a tongue of the user from obstructing an airway of the user.
